(19) United States
(12) Patent Application Publication
(10) Pub. No.: US 2014/0275619 A1
(43) Pub. Date: Sep. 18, 2014

(54) Process for Producing Acetic Acid and/or Ethanol By Methane Oxidation

(71) Applicant: CELANESE INTERNATIONAL CORPORATION, Irving, TX (US)

(72) Inventors: Wensheng Chen, Friendswood, TX (US); Grant Proulx, Friendswood, TX (US)

(73) Assignee: CELANESE INTERNATIONAL CORPORATION, Irving, TX (US)

(21) Appl. No.: 13/835,442

(22) Filed: Mar. 15, 2013

(51) Int. Cl. C07C 51/16; C07C 29/149

(52) U.S. Cl. CPC C07C 51/16; C07C 29/149
USPC 562/512.2; 568/885

(57) ABSTRACT

The process relates to producing acetic acid and/or ethanol from methane. Oxidative coupling of methane (OCM) may produce a mixture of ethane and ethylene that is further oxidized to acetic acid. The ethylene may be hydrated to form ethanol. In addition, the acetic acid may be hydrogenated to produce ethanol.

### FIELD OF THE INVENTION

The present invention relates generally to processes for producing acetic acid and/or ethanol by integrating an oxidative coupling of methane reactor with an ethane oxidation reactor. This allows acetic acid and/or ethanol to be produced from methane.

### BACKGROUND OF THE INVENTION

Methane, due to its large worldwide reserves, has been considered desirable to develop processes to convert methane to more valuable chemical products. Methane is a colorless, odorless gas that is typically found in natural gas. Methods for oxidative coupling of methane (OCM) to high hydrocarbons are typically carried out using metal oxide-based catalysts as described in U.S. Pat. Nos. 6,096,934; 5,354,936; 5,212,139; 5,051,390; 4,939,310; 4,547,608; and 4,499,322. Among the known conversion processes, catalysts based on manganese, magnesium and lead using alkali metal promoters have given good results in synthesis of methane to obtain ethane and ethylene type products. Among these catalysts used in such processes, catalysts based upon oxides of manganese, tin, iridium, germanium, lead, antimony and bismuth have been found particularly useful as catalysts in the OCM process. The reactor effluent of the OCM reactor comprises ethane and ethylene.

U.S. Pat. No. 7,368,598 describes processes for direct, selective conversion of methane to acetic acid in a single step. Methane is reacted with a Pt, Pd, Rh, Ru, Os or Ir based catalyst and an oxidant in an acid media, such as H₂SO₄, O₂, H₂SeO₄, TeO₃, H₂O₂, trifluoroacetic, triflic acid or an ionic liquid.

U.S. Pat. No. 7,009,074 describes producing acetic acid by oxidation of methane with an oxygen-containing gas in the presence of an acid selected from concentrated sulfuric acid and fuming sulfuric acid, a palladium-containing catalyst and a promoter, preferably a copper or iron salt. The addition of a promoter and O₂ to a system that includes a palladium-containing catalyst such as PdCl₂ increases the rate of acetic acid formation from methane and, in addition, inhibits the precipitation of Pd black.

US Pub. No. 2003/0158440 discloses a method for the selective production of acetic acid from a gas-phase feed of ethane, ethylene, or mixtures thereof and oxygen at elevated temperatures. The gas-phase feed is brought into contact with a catalyst containing the elements Mo, Pd, and additional metals, such as Nb. The selectivity of the oxidative reaction of ethane and/or ethylene to give acetic acid is greater than 70 mol. %.

U.S. Pat. No. 5,162,578 discloses a process for the higher selective production of acetic acid by the catalytic oxidation with oxygen of ethane, or ethylene, or mixtures thereof, in contact with a mixed catalyst composition containing (A) a calcined mixed oxides catalyst of the formula: MoₓV_{y}Z_{z}. Z represents nothing or is one or more of the metals from the list Nb, Sb, Li, Sc, Na, Be, Mg, Ca, Sr, Ba, Ti, Zr, Hf, Y, Ta, Cr, Fe, Co, Ni, Ce, La, Zn, Cd, Hg, Al, Tl, Pb, As, Bi, Te, U and Wand (B) an ethylene hydration catalyst and/or an ethylene oxidation catalyst.

The need remains for processes for making ethanol from available industrial sources.

### SUMMARY OF THE INVENTION

In a first embodiment, the present invention is directed to a process for making acetic acid, comprising contacting a methane-containing gas and an oxygen-containing gas in a first reactor with a first catalyst for oxidatively coupling methane to form an effluent comprising ethane and ethylene, and oxidizing at least a portion of the effluent in a second reactor with a second catalyst to form a crude product stream comprising acetic acid. The effluent may have an ethane to ethylene molar ratio from 0.5:1 to 2:1 and may further comprise one or more of methane, water, and carbon oxides. In one embodiment, methane may be recovered from the crude product or effluent and recycled to the first reactor. The process may further comprise hydrating, either through direct or indirect hydration, at least 10% of the ethylene from the vapor phase stream in a hydration zone into ethanol. The first reactor is an OCM reactor and comprises manganese oxide catalyst incorporating at least one of the elements selected from the group consisting of tin, titanium, tungsten, tautalum, silicon, germanium, lead, phosphorus, arsenic, antimony, boron, gallium, indium, a lanthanide and an actinide. In another embodiment, the catalyst of the first reactor comprises an alkali and/or alkaline earth metal oxide. The second catalyst may be more selective for acetic acid than ethylene and may comprise at least molybdenum, vanadium, and preferably palladium.

In a second embodiment, the present invention is directed to a process for making acetic acid and ethanol, comprising contacting a methane-containing gas and an oxygen-containing gas in a first reactor with a first catalyst for oxidatively coupling methane to form an effluent comprising ethane and ethylene; oxidizing at least a portion of the effluent in a second reactor with a second catalyst to form a crude product stream comprising acetic acid and ethylene, separating the crude product stream into an overhead stream and a bottoms stream, wherein the overhead stream comprises a majority of the ethane, ethylene and carbon dioxide from the crude product stream, and wherein the bottoms stream comprises a majority of the acetic acid and water from the crude product stream; and hydrating at least 10% of the ethylene from the overhead stream in a hydration zone into ethanol.

In a third embodiment, the present invention is directed to a process for making acetic acid, comprising contacting a methane-containing gas and an oxygen-containing gas in a first reactor with a first catalyst for oxidatively coupling methane to form an effluent comprising ethane and ethylene, separating the effluent into a methane recycle stream and an ethane and ethylene mixture stream, recycling the methane recycle stream to the first reactor, directing the ethane and ethylene mixture stream to the second reactor and oxidizing at least a portion of the effluent in a second reactor with a second catalyst to form a crude product stream comprising acetic acid. The process may further make ethanol by separating the crude product stream into an overhead stream and a bottoms stream, wherein the overhead stream comprises a majority of the ethane, ethylene and carbon dioxide from the crude product stream, and wherein the bottoms stream comprises a majority of the acetic acid and water from the crude product stream, and hydrating at least a portion of the overhead stream comprising ethylene. In another embodiment, the acetic acid may be hydrogenated to produce ethanol.

### BRIEF DESCRIPTION OF THE DRAWINGS

FIG. 1 is a representation of an integrated process for oxidative coupling of methane (OCM) to produce a mixture of ethane and ethylene and oxidizing the mixture to produce acetic acid in accordance with an embodiment of the present invention.

FIG. 2 is a representation of an integrated process for OCM and separating the effluent to recover a mixture of ethane and ethylene and oxidizing the mixture to produce acetic acid in accordance with an embodiment of the present invention.

FIG. 3 is a representation of an integrated process for OCM to produce a mixture of ethane and ethylene, oxidizing the mixture to produce acetic acid, and indirect hydrating of the ethylene to produce ethanol in accordance with an embodiment of the present invention.

FIG. 4 is a representation of an integrated process for OCM and separating the effluent to recover a mixture of ethane and ethylene, oxidizing the mixture to produce acetic acid, and indirect hydrating of the ethylene to produce ethanol in accordance with an embodiment of the present invention.

### DETAILED DESCRIPTION OF THE INVENTION

### A. Introduction

In general, the process relates to producing acetic acid and/or ethanol from methane. In one embodiment, oxidative coupling of methane (OCM) may produce a mixture of ethane and ethylene that is further oxidized to acetic acid. The ethylene may be hydrated to form ethanol. In addition, the acetic acid may be hydrogenated to produce ethanol. This allows a producer to make a variety of products from methane including acetic acid, ethanol, ethylene, and combinations thereof. The flexibility to produce a variety of products allows a producer to remain competitive as market demand fluctuates.

One problem with OCM is that the reactor effluent comprises a mixture of ethylene and ethane. None of the commercially available catalysts can selectivity produce ethylene without ethane. The resulting mixture is particular difficult to separate and ethane or ethylene cannot be readily recovered. To solve the problem with separating the mixture of ethylene and ethane, the present invention feeds the mixture to a secondary oxidation process, namely ethane oxidation, to produce acetic acid and/or ethylene. In some embodiments, the ethylene being fed may be converted to acetic acid. In one embodiment, the reactor effluent from the OCM reactor is directly fed to the ethane oxidation step. In another embodiment, methane may be removed prior to the ethane oxidation step. Separating acetic acid from ethylene requires less capital and energy than separating ethane and ethylene. In addition, ethanol may be produced from ethylene or acetic acid further facilitating product separation.

The present invention comprises at least one OCM reactor and at least one ethane oxidation reactor. Additional reactors, such as hydration reactors, hydrogenation reactors, and/or methanation reactors, may be added in various combinations as described herein to form an integrated process that is capable of producing multiple products. Various combinations include but are not limited to the following. An exemplary integrated process may comprise an OCM reactor, ethane oxidation reactor, and hydration reactor. The order of the reactors may vary, but typically the OCM reactor is the initial reactor. Another exemplary integrated process may comprise an OCM reactor, ethane oxidation reactor, and hydrogenation reactor. Another exemplary integrated process may comprise an OCM reactor, ethane oxidation reactor, hydration reactor, and hydrogenation reactor. Another exemplary integrated process may comprise an OCM reactor, ethane oxidation reactor, methanation reactor and hydration reactor. Yet another exemplary integrated process may comprise an OCM reactor, ethane oxidation reactor, methanation reactor, hydrogenation reactor and hydration reactor.

### B. Methane Oxidization

In the first oxidation reaction, namely OCM, a methane-containing gas and an oxygen-containing gas are fed to a reactor to produce a mixture of ethane and ethylene. Regardless of the source, the methane to oxygen molar ratio may be from 1.1:1 to 50:1, e.g., from 2:1 to 10:1. Selectivity to ethane and ethylene may be improved with higher methane ratios. In addition to the reactants, the gases may also comprise one or more inerts selected from the group consisting of argon, helium, nitrogen, carbon dioxide and steam.

Methane is the lowest molecular weight, and simplest in structure, of the hydrocarbons. The source of methane is typically natural gas, the composition of which can vary significantly from source to source. Natural gas may be directly used or purified as needed. Due to impurities in natural gas, such as hydrogen sulfide, carbon dioxide, nitrogen, water, and other hydrocarbons, it may be desirable to purify the natural gas. Without being bound by theory, some of the impurities, such as hydrogen sulfide or methyl mercaptan, might interfere with oxidation catalysts in either the OCM or ethane oxidation reactor. Other sources for the methane-containing gas may include renewable sources such as landfills, farms, biogas from fermentation, or fossil fuels such as oil accompanying gases, coal gas, and gas hydrates.

It is preferred to add oxygen-containing gas in a concentration range outside of the explosion limits under reaction conditions. Oxygen-containing gas may refer to air, oxygen enriched air, or pure oxygen. Oxygen may be separated from air by low temperature processing and oxygen may be concentrated by means of gas separation membranes that can produce pure oxygen or oxygen enriched air. Air refers to a mixture of gases with a composition approximately identical to the native composition of gases taken from the ambient surroundings. Air includes approximately 78 vol. % nitrogen, approximately 21 vol. % oxygen, approximately 1 vol. % argon, and approximately 0.04 vol. % carbon dioxide. Oxygen enriched air generally refers to a mixture of gases with a composition comprising more oxygen than is present in air. Oxygen enriched air may refer to an oxygen-containing gas that contains less nitrogen, argon, and/or carbon dioxide than air. For example, a mixture of air and pure oxygen may also be referred to as oxygen enriched air.

The catalyst for the OCM reaction may vary and preferably is a catalyst that has a sufficient conversion of methane. This includes the catalysts described in U.S. Pat. Nos. 6,096,934; 5,354,936; 5,212,139; 5,051,390; 4,939,310; 4,547,608; and 4,499,322, the entire contents and disclosures of which are hereby incorporated by reference. The selectivity to ethane and/or ethylene is generally favored over other components.

In one embodiment, the catalyst comprises an alkali and/or alkaline earth metal oxide, or optionally lanthanide oxides, based catalysts. The most preferred alkali and/or alkaline earth metal oxide based catalysts include oxides of Li, Mg, Ba, Ca, La, and combinations thereof.

In another embodiment, the catalyst comprises a manganese oxide incorporating at least one of the elements tin, titanium, tungsten, tautalum, silicon, germanium, lead, phosphorus, arsenic, antimony, boron, gallium, indium, a lanthanide or an actinide. The catalyst may also be promoted by either an alkali or alkaline earth metal, for example sodium. The catalyst may be employed in the form of a fixed bed, a fluidised bed, a particulate bed or a recirculating bed, or in any other convenient form. The catalyst may be prepared by suitable methods such as those described in U.S. Pat. No. 5,212,139, the entire contents and disclosures of which is hereby incorporated by reference.

In another embodiment, the catalyst may be based on a reducible metal oxide selected from oxides of Mn, Sn, In, Ge, Pb, Sb, Bi, Pr, Tb, Ce, Fe, and Ru, supported on silica and promoted by addition of tungsten, an alkali metal, and niobium or an oxide thereof, as described in U.S. Pat. No. 7,902,113, the entire contents and disclosure of which is hereby incorporated by reference. Niobium may be present in the catalyst in an amount from 0.5 to 5 wt. %, based on the total amount of the catalyst, e.g., from 1.2 to 4 wt. %. The atomic ratio of reducible metal to tungsten is optionally from 1.8 to 3.5. The atomic ratio of the reducible metal oxide to alkaline metal may be in the range from 0.1 to 0.6. The atomic ratio of the promoter to reducible metal is preferably from 0.01 to 0.8, e.g., from 0.005 to 0.03. Optionally, this catalyst may further comprise a metal selected from Eu, Y and Nd.

In another embodiment, the catalyst for the OCM reaction may comprise SnBaTiO₃, as described in U.S. Pat. No. 7,250,543, the entire contents and disclosure of which is hereby incorporated by reference. This catalyst may be particularly selective to ethylene.

The methane-containing gas and oxygen-containing gas are continuously supplied to an OCM reactor containing the catalysts, and the reaction is preferably conducted at a temperature from 500° C. to 1000° C., e.g., from 600° C. to 900° C. or from 700° C. to 850° C. The pressure may be in the range from 0 to 10 MPa, e.g., from 0.1 to 3 MPa, or under atmospheric pressure. The gas is preferably supplied to the reactor at a gas hourly space velocity measured at standard temperature and pressure in the range from 100 to 100,000 hr⁻¹, and more preferably from 300 to 12,000 hr⁻¹.

The conversion of methane may be at least 10%, e.g., at least 30%. In terms of ranges the conversion of methane may be from 10% to 80%, e.g., from 20 to 60%. Oxygen conversion is generally greater than 90% or greater than 97%. For purposes of the present invention, the term “conversion” refers to the amount of reactant, whichever is specified, in the feed to the reactor that is converted to a compound other than the reactant. Conversion is expressed as a percentage based on the specific reactant in the feed to the reactor.

The selectivity of methane to ethane may be at least 5%, e.g., at least 25%. The selectivity to ethylene may be at least 5%, e.g., at least 25%. In terms of ranges the selectivity to ethane and/or ethylene may be from 5% to 60%, e.g., from 10 to 50%. Selectivity is expressed as a mole percent based on converted reactant, such as methane. It should be understood that each compound, ethane and ethylene, converted from methane has an independent selectivity and that selectivity is independent from conversion. Preferred OCM processes also have low selectivity to undesirable products, such as carbon oxides and higher hydrocarbons containing three or more carbon atoms. The selectivity to these undesirable products preferably is less than 50%, e.g., less than 30% or less than 20%.

The effluent from the OCM reactor comprises methane, ethane, ethylene, water, and carbon oxides, and heavy hydrocarbons. For purposes of the present invention, carbon oxides includes carbon dioxide and carbon monoxide, as well as other carbon oxides. The term “heavy hydrocarbons” refers to alkanes and alkenes having three or more carbon atoms. The ethane to ethylene molar ratio in the effluent may be from 0.5:1 to 2:1, e.g., 0.6:1 to 1.5:1. In one embodiment, the effluent may comprise from 35 to 85 vol. % methane, e.g., from 35 to 50 vol. %. The effluent may also comprise from 10 to 45 vol. % ethane, e.g., from 10 to 35 vol. % or from 20 to 30 vol. %. The effluent may also comprise from 10 to 45 vol. % ethylene, e.g., from 20 to 30 vol. %. In one embodiment, the effluent may comprise from 10 to 50 vol. % water, e.g., from 10 to 40 vol. %. The effluent may also comprise from 3 to 30 vol. % carbon oxides, e.g., from 5 to 25 vol. %. The effluent may further comprise from 0 to 10 vol. % heavy hydrocarbons, e.g., from 0 to 8 vol. %. The heavy hydrocarbons may also be converted to their corresponding acids in the ethane oxidation reactor.

In one embodiment, the effluent from the OCM reactor is directly fed to the ethane oxidation reactor. In another embodiment, methane may be separated and recovered from the effluent prior to introducing the effluent into the ethane oxidation reactor.

### C. Ethane Oxidation

Catalysts for oxidizing the mixture of ethane and ethylene may include those that are capable of producing acetic acid or ethylene. Because the effluent from the OCM reactor may be directly fed to ethane oxidation, the effluent may also contain methane. The catalysts suitable for ethane oxidation should not be deactivated in the presence of methane. In addition, preferably the catalyst for oxidizing the mixture of ethane and ethylene are selective for acetic acid production. In some embodiments, the ethylene in the mixture may pass through the ethane oxidation reactor without being converted to another compound. In other embodiments, less than 5% of the ethylene in the mixture may be converted to acetic acid when passing through the ethane oxidation reactor.

Catalysts for oxidizing ethane to acetic acid, and catalysts for oxydehydrogenation reactions, i.e., which primarily produce ethylene from ethane, include catalysts containing molybdenum and/or vanadium, including oxides thereof. Suitable catalysts are described in U.S. Pat. Nos. 4,250,346; 4,524,236; 4,568,790; 6,034,270; 6,274,765; 6,399,816; 6,906,221; 7,015,355; and 7,081,549, each of which is incorporated herein by reference in its entirety. Exemplary catalysts may comprise molybdenum and at least one metal selected from the group consisting of niobium, vanadium, titanium, tungsten, tantalum, palladium, rhenium, lead, antimony, iron, bismuth, and silicon. In particular, the catalyst that may be selective to ethylene may be an oxide or carbide catalyst comprising Mo, V, Nb, Sb, or combinations thereof. An additional metal may also be present in the oxide or carbide catalyst. Another oxide or carbide catalyst may be more selective to acetic acid and may comprise Mo, Pd, Re, and at least one other metal. The oxide or carbide catalysts may be on a support that is selected from the group consisting of silica, alumina, silica-alumina, silicon carbide, titania, zirconia, and mixtures thereof. Other inorganic oxides or carbides may also be used. Preferred support materials have a surface area of less than 250 m²/g. The metals or oxides thereof may be impregnated on the support using incipient wetness techniques. In one embodiment, acetic acid selectivity is preferred over ethylene with a noble metal, e.g., Pd, containing metal oxide catalyst in the presence of steam as co-feed. Although, the catalyst may favor one species over the other, some of the catalysts may also produce a mixture of ethylene and acetic acid as described in U.S. Pat. No. 4,250,346, which is incorporated herein by reference in its entirety.

In one embodiment, the catalyst for the ethane oxidation reaction is selective for the preparation of acetic acid from an ethane and ethylene mixture, and the catalyst comprises an oxide-based catalyst having the formula:

MoₐPd_{b}Re_{c}X_{d}Yₑ

Wherein X may be Cr, Mn, Nb, B, Ta, Ti, V and/or W. In one embodiment, X is Nb, V and/or W. Y may be Bi, Ce, Co, Cu, Te, Fe, Li, K, Na, Rb, Be, Mg, Ca, Sr, Ba, Ni, P, Pb, Sb, Si, Sn, Tl and/or U, in particular Ca, Sb, Te and/or Li. The gram ratios of a, b, c, d and e are as follows: a is 1, b is from 0.0001 to 0.5, c>0 and preferably is from 0.25 to 1, d is from 0.05 to 2 and e is from 0 to 3. When X and Y are a plurality of different elements, the indices d and e can likewise assume a plurality of different values. Examples of such oxide-based catalyst compositions include:

Mo₁Pd_{0.01}Re_{0.7}V_{0.7}Nb_{0.2}Sb_{0.1}Ca_{0.05}

Mo₁Pd_{0.02}Re_{0.7}V_{0.7}Nb_{0.2}Sb_{0.1}Ca_{0.05}

Mo₁Pd_{0.02}Re_{0.5}V_{0.5}Nb_{0.5}Sb_{0.1}

Mo₁Pd_{0.02}Re_{0.7}V_{0.5}Te_{0.5}

Mo₁Pd_{0.02}Re_{0.7}W_{0.2}V_{0.7}Nb_{0.2}Sb_{0.1}

Mo₁Pd_{0.00075}V_{0.55}Nb_{0.9}Sb_{0.1}Ca_{0.1}

The effluent from the OCM reactor is reacted with a second oxygen-containing gas in the form of air, oxygen enriched air or pure oxygen as described above. Diluents are not required for ethane oxidation, but may be present if air or oxygen enriched air is used. The oxidation and/or oxidative dehydrogenation reaction is preferably carried out in the presence of added water (steam) in the gas phase at a temperature less than 550° C., e.g., from 200° C. to 400° C. The reaction pressure may be from 0.1 to 5 MPa, e.g., from 1 to 2 MPa. In one embodiment, one mole of ethane may be reacted with 0.01 to 0.5 moles of molecular oxygen. Higher oxygen contents are preferred, since the achievable ethane conversion and thus the yield of oxygenates may be higher. The concentration of oxygen in the ethane reactor is generally less than 40 mol. %, e.g., less than 35 mol. % or less than 30 mol. %. It is preferred to add oxygen or gas containing molecular oxygen in a concentration range outside of the flammability limits under reaction conditions. However, it is also possible to set the ethane to oxygen molar ratio within the flammability limits by controlling the feed of the oxygen to the ethane oxidation reactor. The oxidation reaction may be carried out in a fluidized bed or a fixed-bed reactor.

In one embodiment, the conversion of ethane may be at least 5%, e.g., at least 15% or at least 50%. Similar to the OCM reactor, oxygen conversion is generally complete and greater than 90% or 95%. In one embodiment, the conversion of ethylene may be zero so that there is not a net decrease or increase in ethylene in the crude product from the ethane oxidation reactor. The selectivity to acetic acid, for the ethane oxidation reaction, is preferably at least 20%, e.g., least 40%, at least 80% or at least 90%. Because acetic acid selectivity is preferred, in one embodiment the selectivity to acetic acid is greater than the selectivity to ethylene. However, in other embodiments, the selectivity to ethylene may be greater than the selectivity to acetic acid. In those embodiments, it may be preferable to convert the ethylene to ethanol through hydration.

### D. Indirect Hydration

Ethylene produced by the OCM reactor and ethane oxidation reactor may be converted to ethanol through a direct or indirect hydration process. The hydration catalyst may be selected from the group consisting of phosphoric acid, sulfuric acid, tungstic acid, heteropoly acid salt and anion ion exchange resin. Typically, sulfuric acid is used for indirect hydration and phosphoric acid is used for direct hydration. For purposes of the present invention, an indirect hydration process is described, but it should be understood that the present invention may be used with direct hydration processes. An indirect hydration process may be integrated more easily with OCM and ethane oxidation due to the lower operating pressures of the OCM and ethane oxidation steps. However, in another embodiment, the OCM and ethane oxidation steps may be integrated with a direct hydration process having an additional compressor.

The indirect hydration process may involve several absorption columns that contain from 94 to 98 wt. % sulfuric acid. The indirect hydration process involves an esterification step to produce an ester followed by hydrolysis. In the esterification step, ethylene is fed to the columns in a countercurrent flow to the sulfuric acid and the columns typically operate at a temperature from 50 to 150° C. and a pressure from 0.1 to 6 MPa. The resulting esters are withdrawn and passed to a hydrolysis zone that typically operates at a temperature from 20° C. to 200° C., e.g., from 70° C. to 100° C., and a pressure from 0.1 to 6 MPa. The hydrolysis step produces the desired alcohol, and a corresponding ether that can be separated. The sulfuric acid is reconstituted from the ester and recycled to react with ethylene. The selectivity to ethanol may be from 60 to 99%, e.g., from 75 to 97%.

### E. Hydrogenation

In some embodiments, ethanol may be obtained by reducing acetic acid with hydrogen in the presence of a catalyst, as described in U.S. Pat. Nos. 7,863,489, and 7,608,744, the entire contents of which are hereby incorporated by reference. The hydrogenation catalyst may comprise platinum, palladium, cobalt, tin, molybdenum, tungsten, or combinations thereof. A hydrogenation catalyst that combines at least platinum and tin is preferred. The hydrogenation catalyst may be supported on a silica support.

The hydrogenation may be carried out in either the liquid phase or vapor phase. Preferably, the reaction is carried out in the vapor phase under the following conditions. The reaction temperature may range from 125° C. to 350° C., e.g., from 200° C. to 325° C., or from 250° C. to 300° C. The pressure may range from 10 kPa to 3000 kPa, e.g., from 50 kPa to 2300 kPa, or from 100 kPa to 2000 kPa. The reactants may be fed to the reactor at a gas hourly space velocity (GHSV) of greater than 500 hr⁻¹, e.g., greater than 1000 hr⁻¹, greater than 2500 hr⁻¹ or even greater than 5000 hr⁻¹. The molar ratio of hydrogen to acetic acid is greater than 2:1, e.g., greater than 4:1 or greater than 8:1.

### F. Recovery/Purification

The present invention will now be described with reference to the schematic diagrams in FIGS. 1-4. It should be understood that these schematics provide a general overview of the process and that additional components may be included with each step. In FIGS. 1-4, process 100 comprises an OCM reactor 102 and ethane oxidation reactor 104. A methane-containing gas 106 and an oxygen-containing gas 108 are fed to OCM reactor 102 to produce an effluent 110, under the conditions described herein. Effluent 110 comprises a mixture of ethylene and ethane. In addition, due to relatively low conversions, effluent 110 also comprises unreacted methane. Carbon oxides, such as carbon dioxide and carbon monoxide, may also be produced in OCM reactor 102.

In FIGS. 1 and 3 effluent 110 is directly fed to ethane oxidation reactor 104 along with a secondary oxygen-containing gas 112. Methane and ethylene in effluent 110 pass through ethane oxidation reactor 104 without reacting. Ethane oxidation reactor 104 produces acetic acid and the crude product in line 116 is directed to a separator 118. Crude product in line 116 comprises methane, ethane, ethylene, acetic acid, water, carbon oxides, and mixtures thereof. In one embodiment, the crude product may comprise from 25 to 50 wt. % methane, 0 to 15 wt. % ethane, 0 to 15 wt. % ethylene, 10 to 40 wt. % acetic acid, 10 to 50 wt. % water, and 1 to 20 wt. % carbon oxides. In some embodiments, the crude product in line 116 may contain no ethane and/or ethylene. Crude product in line 116 may be a vapor that exits ethane oxidation reactor 104 at a temperature from 450° C. to 600° C.

Separator 118 forms an overhead stream 120 comprising ethylene and methane and a bottoms stream 122 comprising acetic acid. In some embodiments, separator 118 may comprise a flasher or a knockout pot. Separator 118 may operate at a temperature from 25° C. to 100° C., e.g., from 25° C. to 50° C. The pressure of separator 118 may be from 100 kPa to 500 kPa, e.g., from 100 kPa to 250 kPa. Optionally, the crude product in line 116 may pass through one or more membranes to separate hydrogen and/or other non-condensable gases.

In another embodiment, crude product in line 116 may be separated using a predehydrating column (not shown) as described in US Pub. No. 2010/0249456, the entire contents and disclosure of which is hereby incorporated by reference. The reactor effluent enters the base of the predehydration tower, and due to the high temperature of the stream, provides most, if not all, of the energy necessary to effectuate the separation of acetic acid from water in the tower. Alternatively, a reboiler at the base of the predehydration tower may be used to provide additional energy input into the tower. The tower may operate at or near the pressure of the ethane oxidation reactor, and would preferably contain 25-35 stages, however the number of stages can vary depending upon the quality of the separation desired. An overhead condensing system cools the overhead gas stream to a temperature below the condensation point of the water vapor, preferably from 100° C. to 120° C., and would provide reflux to the predehydration tower. An overhead liquid water product is recovered having very low levels of acetic acid therein, preferably less than 1 wt. %, allowing for that stream to be disposed of biologically. The bottoms stream from the predehydration tower is crude acetic acid with much lower water content, preferably less than 10% water, than had the stream been processed in a conventional stripper. The gaseous top stream from the predehydration tower is routed to a fixed bed CO converter followed by a processing step that removes the carbon oxides from the top stream. This purified stream is then recycled to the ethane oxidation reactor for further conversion into acetic acid.

Bottoms stream 122 comprises a majority of the acetic acid and water from the crude product stream. For example, bottoms stream 122 comprises 25 wt. % to 75 wt. % acetic acid, e.g., 40 wt. % to 70 wt. %, and 25 wt. % to 75 wt. % water, e.g., 30 wt. % to 60 wt. %. In one embodiment, water may be removed from bottom stream 122 to obtain a glacial acetic acid product. In another, the bottoms stream 122 may be fed to a hydrogenation reactor to produce ethanol. Alternatively, the glacial acetic acid product may also be fed to a hydrogenation reactor to produce ethanol.

Overhead stream 120 comprises a majority of the methane, ethane, ethylene and carbon oxides from the crude product stream 116. Overhead stream 120 comprises 0 wt. % to 35 wt. % ethylene, e.g., 0 wt. % to 20 wt. %, 5 wt. % to 35 wt. % ethane, e.g., 5 wt. % to 20 wt. %, 20 wt. % to 70 wt. % methane, e.g., 20 wt. % to 50 wt. %, and 5 wt. % to 20 wt. % carbon oxides, e.g., 5 wt. % to 15 wt. %. Overhead stream 120 is passed to an adsorption zone 124 containing an adsorbent capable of selectively adsorbing the carbon oxides so that a majority of the carbon oxides are removed. In one embodiment, adsorption zone 124 may comprise a scrubber. Suitable adsorbents include lanthanum oxide with at least one alkali metal hydroxide or oxide, such as lithium hydroxide, alkali and alkaline earth exchanged X- and Y-type zeolites and other suitable zeolitic materials possessing a large adsorption equilibrium constant for carbon oxides. Overhead stream 120 contacts the adsorbents and the carbon oxides are selectively adsorbed and separated from the methane, ethane, and ethylene. Adsorption zone 124 may be operated in a swing bed, moving bed, or simulated moving bed mode at temperatures in the range from 0° C. to 400° C. and pressures in the range from 0.1 MPa to 3.5 MPa. The carbon oxides are separated from overhead stream 120 and are immobilized by a solid phase. The remaining vapor phase is removed in line 126 from adsorption zone 124. Vapor phase in line 126 comprises methane, ethane, and ethylene. In one embodiment, the carbon oxides are desorbed from the adsorbent using hot hydrogen, up to 1000° C., to generate a stream containing hydrogen and carbon oxides.

Optionally, the carbon oxides and hydrogen from adsorption zone 124 may be directed to a methanation reactor (not shown). The methanation reactor contains a catalyst effective in catalyzing the methanation reaction of carbon monoxide or carbon dioxide and hydrogen to form methane and water. The methane may be returned to OCM reactor 102. The methanation catalyst comprises a bifunctional catalyst comprising a metal component, particularly a transition metal or compound thereof, on an inorganic support. Suitable metal components comprise copper, iron, vanadium, chromium, zinc, gallium, nickel, cobalt, molybdenum, ruthenium, rhodium, palladium, silver, rhenium, tungsten, iridium, platinum, gold, gallium and combinations and compounds thereof. The inorganic support may be an amorphous material, such as silica, alumina or silica-alumina, or like those listed for the dehydroaromatization catalyst. In addition, the inorganic support may be a crystalline material, such as a microporous or mesoporous crystalline material. Suitable porous crystalline materials include the aluminosilicates, aluminophosphates and silicoaluminophosphates. Examples of suitable catalysts are ruthenium on alumina, platinum-ruthenium on alumina, and Raney nickel on alumina.

The optional methanation reactor is typically operated in a fixed bed mode but may be operated in a moving bed, swing bed, or simulated moving bed mode. The reaction temperature may range from 125° C. to 350° C., e.g., from 200° C. to 325° C., from 225° C. to 300° C., or from 250° C. to 300° C. The pressure may range from 10 kPa to 3000 kPa, e.g., from 50 kPa to 2300 kPa, or from 100 kPa to 1500 kPa. The methane and water produced in the methanation reactor are removed in an effluent which is processed to separate the methane from the water. Suitable separation processes are well known in the art and include such techniques as adsorption and distillation. The preferred technique is adsorption drying using 4° A molecular sieve. The dried methane is then recycled to OCM reactor 102. The use of a methanation reactor allows conservation of carbon to improve overall efficiency of the OCM reactor.

Vapor phase in line 126 may be separated to recover methane that may be returned to OCM reactor 102 and a stream of ethylene and/or ethane that may be returned to ethane oxidation reactor 104. In one embodiment vapor phase in line 126 is not condensed prior to being returned to the respective reactor. In FIG. 1, vapor phase in line 126 passes through compressor 128 and is pumped at a pressure from 3 MPa to 4.1 MPa and is kept at a low temperature, e.g., from −40° C. to −80° C., into a demethanizer column 130. Demethanizer column 130 produces an overhead stream 132 comprising a majority of the methane and a bottoms stream 134 comprising a majority of ethane from the feed to the demethanizer column 130. Overhead stream 132 comprises 70 wt. % to 99 wt. % methane, 0 wt. % to 30 wt. % ethane, 0 wt. % to 30 wt. % ethylene. Bottoms stream 134 comprises 50 wt. % to 99 wt. % ethane, and 0 wt. % to 20 wt. % ethylene. In one embodiment, bottoms stream 134 does not contain any methanol.

In FIG. 3, upstream of demethanizer column 130, there is a hydration zone 140. Hydration zone 140 may be a direct hydration reactor or an indirect hydration reactor. Depending on the type of hydration, there may be multiple absorbers, columns, reactors, and hydrolysis units. Vapor phase in line 126 which comprises ethylene is fed to hydration zone 140 to convert the ethylene to ethanol. In FIG. 3, methane is not separated until after hydration zone 140 and thus methane also is fed to hydration zone 140. However, methane preferably does not react in hydration zone 140. When direct hydration is used, vapor phase in line 126 may be compressed as needed. A separate water stream (not shown) may also be fed to hydration zone 140. In one embodiment, at least 10% of the ethylene in vapor phase in line 126 is converted to ethanol. The light impurities, namely methane and ethane, are recovered in line 142 and forwarded to a demethanizer column 130. Having hydration zone 140 upstream of demethanizer column 130 may reduce the size of demethanizer column 130 due to less ethylene passing into the demethanizer column 130. Ethanol is recovered in line 144 and may be dried to produce anhydrous ethanol. Due to the decrease in ethylene concentration in demethanizer column 130, in FIG. 3 demethanizer column 130 produces an overhead stream 132 and a bottoms ethane stream 138. Ethane stream 138 is enriched in ethane compared with bottoms stream 134, and ethane stream 138 may be fed to ethane oxidation reactor 104.

In one alternative embodiment, the hydration zone 140 may be placed between the OCM reactor 102 and ethane oxidation reactor 104. This allows the ethylene produced from the OCM reactor 102 to be converted to ethanol prior to converting the ethane to acetic acid. In addition, this appoarch reduces the volume of reactants flowing into ethane oxidation reactor 104 because there is less ethylene in the feed thereto.

In FIGS. 2 and 4 methane is separated before ethane oxidation reactor 104 using a splitter column 150. Effluent 110 passes through compressor 152 and is fed into splitter column 150. Splitter column separates effluent 110 into three components: methane recycle stream 154, ethane and ethylene mixture stream 156, and a carbon oxide stream 158. Methane recycle stream 154 is returned to OCM reactor 102. Ethane and ethylene mixture stream 156 is deficient with respect to methane as compared to effluent 110 and is fed to ethane oxidation reactor 104. Carbon oxide stream 158 may be forwarded to adsorption zone 124. The vapor phase in line 126 from adsorption zone 124 may be returned to ethane oxidation reactor 104 without having to further separate methane.

Because methane is removed upstream of ethane oxidation reactor 104 in FIG. 4, little or no methane is fed to ethane oxidation reactor 104 or hydration zone 140.

Each column shown in the figures may comprise one or more distillation columns capable of performing the desired separation and/or purification steps. Each column preferably comprises a tray column having from 1 to 150 trays, e.g., from 10 to 100 trays, from 20 to 95 trays or from 30 to 75 trays. The trays may be sieve trays, fixed valve trays, movable valve trays, or any other suitable design known in the art. In other embodiments, a packed column may be used. For packed columns, structured packing or random packing may be employed. The trays or packing may be arranged in one continuous column or they may be arranged in two or more columns such that the vapor from the first section enters the second section while the liquid from the second section enters the first section.

The associated condensers and liquid separation vessels that may be employed with each of the distillation columns may be of any conventional design and are simplified in the figures. Heat may be supplied to the base of each column or to a circulating bottom stream through a heat exchanger or reboiler. Other types of reboilers, such as internal reboilers, may also be used. The heat that is provided to the reboilers may be derived from any heat generated during the process that is integrated with the reboilers or from an external source such as another heat generating chemical process or a boiler. In addition the components shown in the figures, additional reactors, flashers, condensers, heating elements, compressors, absorbers, scrubbers, and other components may be used in various embodiments of the present invention. As will be recognized by those skilled in the art, various condensers, pumps, compressors, reboilers, drums, valves, connectors, separation vessels, etc., normally employed in carrying out chemical processes may also be combined and employed in the processes of the present invention.

The temperatures and pressures employed in the columns may vary. As a practical matter, pressures from 10 kPa to 3000 kPa will generally be employed in these columns although subatmospheric pressures or superatmospheric pressures may also be employed. Temperatures within the various zones will normally range between the boiling points of the composition removed as the distillate and the composition removed as the residue. As will be recognized by those skilled in the art, the temperature at a given location in an operating distillation column is dependent on the composition of the material at that location and the pressure of column. In addition, feed rates may vary depending on the size of the production process and, if described, may be generically referred to in terms of feed weight ratios.

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

### EXAMPLE

A stream having 42.2 mol. % ethane, 10.5 mol. % methane, 19 mol. % oxygen and 28.2 mol. % nitrogen was fed to an ethane oxidation reactor. The ethane oxidation reactor contained a catalyst having the formula Mo₁Pd_{0.00075}V_{0.55}Nb_{0.9}Sb_{0.1}Ca_{0.1}. The reaction was carried out at a temperature of 320° C. and a pressure of 1.48 MPa. The contact time was 8.77 seconds. The ethane conversion was 33.53% and oxygen conversion was 97.11%. The selectivity to ethylene was 51.02%, and acetic acid selectivity was 25.91%. The selectivity to carbon oxides was 6.93% carbon monoxide and 9.25% carbon dioxide. Overall the STY production of ethylene was 246.52 g/L/hr and acetic acid was 270.52 g/L/hr.

This example demonstrates that addition of methane to the feed can be processed in ethane oxidation reactor and give a gas product stream with both ethylene concentration and overall pressure suitable for integration with indirect ethylene hydration to ethanol.

We claim:

1. A process for making acetic acid, comprising:
contacting a methane-containing gas and an oxygen-containing gas in a first reactor with a first catalyst for oxidatively coupling methane to form an effluent comprising ethane and ethylene; and
oxidizing at least a portion of the effluent in a second reactor with a second catalyst to form a crude product stream comprising acetic acid.

2. The process of claim 1, wherein the effluent has an ethane to ethylene molar ratio from 0.5:1 to 2:1.

3. The process of claim 1, wherein the effluent further comprises methane, water and carbon oxides.

4. The process of claim 1, wherein methane is recovered from the crude product stream and recycled to the first reactor.

5. The process of claim 1, wherein methane is recovered from the effluent and recycled to the first reactor.

6. The process of claim 1, further comprising:
separating the crude product stream into an overhead stream and a bottoms stream, wherein the overhead stream comprises a majority of the methane, ethane, ethylene and carbon oxides from the crude product stream, and wherein the bottoms stream comprises a majority of the acetic acid and water from the crude product stream.

7. The process of claim 6, further comprising:
feeding at least a portion of the bottoms stream to a third reactor along with hydrogen to convert the acetic acid to ethanol.

8. The process of claim 6, further comprising:
removing a majority of the carbon oxides from the overhead stream in an adsorption zone to form a vapor phase stream.

9. The process of claim 8, further comprising:
hydrating at least 10% of the ethylene from the vapor phase stream in a hydration zone into ethanol; and
recovering an ethanol product stream from the hydration zone.

10. The process of claim 9, further comprising:
recovering a light impurity stream comprising methane and ethane from the hydration zone; and
separating the light impurity stream in a demethanizer column into an second overhead stream comprising a majority of the methane from the light impurity stream, and a second bottoms stream comprising a majority of the ethane from the light impurity stream.

11. The process of claim 10, further comprising:
recycling at least a portion of the second overhead stream to the first reactor; and
recycling at least a portion of the second bottoms stream to the second reactor.

12. The process of claim 1, wherein the first catalyst comprises manganese oxide incorporating at least one of the elements selected from the group consisting of tin, titanium, tungsten, tautalum, silicon, germanium, lead, phosphorus, arsenic, antimony, boron, gallium, indium, a lanthanide and an actinide.

13. The process of claim 1, wherein the second catalyst comprises molybdenum and vanadium.

14. The process of claim 1, wherein the second catalyst is more selective for acetic acid than ethylene.

15. The process of claim 1, further comprising:
separating the effluent into a methane recycle stream and an ethane and ethylene mixture stream;
recycling the methane recycle stream to the first reactor; and
directing the ethane and ethylene mixture stream to the second reactor.

16. The process of claim 15, further comprising:
separating the crude product stream into an overhead stream and a bottoms stream, wherein the overhead stream comprises a majority of the ethane, ethylene and carbon dioxide from the crude product stream, and wherein the bottoms stream comprises a majority of the acetic acid and water from the crude product stream.

17. The process of claim 16, further comprising:
removing a majority of the carbon dioxide from the overhead stream in an adsorption zone to form a vapor phase stream.

18. The process of claim 17, further comprising:
hydrating at least 10% of the ethylene from the vapor phase stream in a hydration zone into ethanol; and
recovering an ethanol product stream from the hydration zone.

19. The process of claim 18, further comprising:
recovering a light impurities stream comprising methane and ethane from the hydration zone; and
recycling at least a portion of the light impurities stream to the first reactor.

20. A process for making acetic acid and ethanol, comprising:
contacting a methane-containing gas and an oxygen-containing gas in a first reactor with a first catalyst for oxidatively coupling methane to form an effluent comprising ethane and ethylene;
oxidizing at least a portion of the effluent in a second reactor with a second catalyst to form a crude product stream comprising acetic acid and ethylene;
separating the crude product stream into an overhead stream and a bottoms stream, wherein the overhead stream comprises a majority of the ethane, ethylene and carbon dioxide from the crude product stream, and wherein the bottoms stream comprises a majority of the acetic acid and water from the crude product stream; and
hydrating at least 10% of the ethylene from the vapor phase stream in a hydration zone into ethanol.
